(19) United States
(12) Patent Application Publication
(10) Pub. No.: US 2007/0179349 A1
(43) Pub. Date: Aug. 2, 2007

(54) System and method for providing goal-oriented patient management based upon comparative population data analysis

(76) Inventors: Kenneth P. Hoyme, Plymouth, MN (US); Howard D. Simms, Shoreview, MN (US); Alan H. Smythe, White Bear Lake, MN (US)

Correspondence Address: CASCADIA INTELLECTUAL PROPERTY, 500 UNION STREET, STE.1005, SEATTLE, WA, 98101, US

(21) Appl. No.: 11/336,741

(22) Filed: Jan. 19, 2006

(51) Int. Cl. A61B 5/00; G06Q 10/00

(52) U.S. Cl. 600/300; 705/2

(57) ABSTRACT

A system and method for providing goal-oriented patient management based upon comparative population data analysis is presented. At least one therapy goal is defined to manage a disease state. A patient population is selected sharing at least one characteristic with an individual patient presenting with indications of the disease state. One or more treatment regimens associated with the patient population are identified as implementing actions under the at least one therapy goal. The implementing actions are followed through one or more quantifiable physiological indications monitored via data sources associated with the patient.

### FIELD OF THE INVENTION

The present invention relates in general to automated patient management and, specifically, to a system and method for providing goal-oriented patient management based upon comparative population data analysis.

### BACKGROUND OF THE INVENTION

In general, implantable medical devices (IMDs) can provide in situ therapy or monitoring under preprogrammed autonomous control. Autonomous control is governed by tunable and fixed control parameters, which are physician-selected to meet therapy goals. IMDs must be periodically interfaced to external devices, such as programmers and patient management devices, for physician follow-up. Physicians assess a patient's condition and follow their progress based on downloaded patient data and lab or clinical tests, such as electrophysiology tests, treadmill stress tests, and blood work, to determine if treatment goals are being met or whether control parameters require reprogramming.

IMD therapy is intended to meet specific therapy goals, such as percentage cardiac pacing, arrhythmia burden, heart rate variability, improved patient symptoms, therapy response, or left ventricular efficiency. A specific form of therapy is selected based upon physician experience and population data. The population data is selected for comparable patient outcomes for patients that started in the same relative health condition as a patient under treatment and is analyzed to find a plan most likely to succeed with the least harm to the patient. Population data can be valuable in providing insight to the potential outcome resulting from IMD therapy for the patient. However, matching patient data to population data is not always practical due to the wide variability in patient profiles, IMD types, and control parameter settings. Moreover, the currency and amount of patient data available for matching to population data is dependent upon the frequency of follow-up, which occur in-clinic once every three to twelve months, or as necessary.

Conventional IMD programming also relies primarily upon population-based data. IMD candidate patients are medically evaluated and broadly characterized using well-known sets of classifications, which include, for example, the New York Heart Association (NYHA) classifications, described in E. Braunwald, ed., “Heart Disease—A Textbook of Cardiovascular Medicine,” Ch. 15, pp. 445-470, W.B. Saunders Co. (5^{th} ed. 1997), the disclosure of which is incorporated by reference. Evaluation can include physical stressors, such as described in Ibid. at Ch. 5, pp. 153-176, pharmacological stressors, as well as sensory, autonomic, or metabolic stressors to establish a diagnosis by determining cardiopulmonary functional capacity and to estimate a treatment prognosis.

IMD programming based upon population-based data, at best, provides a starting point that requires further refinement to tailor therapy to a recipient patient. Classifications are helpful as an aid to providing an initial set of parameters and can be supplemented by treatment strategies obtained through evaluation of patient data collected in patient population databases.

U.S. Pat. No. 6,669,631, issued on Dec. 30, 2003 to Norris et al., discloses deep computing applications in medical device systems. The system includes a medical information network with a centralized database that accepts !MD-developed patient data and patient data derived from other sources. Deep computing technologies are applied to the assembled body of data to develop and provide patient-specific information to a healthcare provider, a patient, or the. patient's family. However, Norris relies on the healthcare provider to make critical healthcare and treatment decisions based on feedback provided by the system through deep computing, rather than making an automated determination on IMD therapy management.

Therefore, there is a need for providing improved remote patient healthcare therapy based upon a broad range of patient population outcomes and closer observations of day-to-day therapy responses within a patient population. Preferably, such an approach would accommodate parametric and physiological data retrieved from internal and external medical devices, as well as repeaters and similar devices, to supplement patient population data and to provide an aid in evaluating treatment goals.

### SUMMARY OF THE INVENTION

A system and method includes formulating a remotely manageable treatment plan to implement a therapy goal based upon a comparative analysis of patient population data. Patient data for those patients sharing at least one characteristic with a patient under treatment is selected from a patient population database and treatment regimens associated with each of the matching patients are identified to provide a set of implementing actions for the patient under treatment. The database stores historical data of patients' responses to various treatment regimens and current health conditions. The implementing actions provide a treatment plan to progress the patient towards the therapy goal and quantifiable physiological indications are monitored through data sources associated with the patient to follow the progress of the treatment plan. As necessary, the treatment plan is reassessed and refined to keep the treatment plan on track.

One embodiment provides a system and method for providing goal-oriented patient management based upon comparative population data analysis. At least one therapy goal is defined to manage a disease state. A patient population is selected sharing at least one characteristic with an individual patient presenting with indications of the disease state. One or more treatment regimens associated with the patient population are identified as implementing actions under the at least one therapy goal. The implementing actions are followed through one or more quantifiable physiological indications monitored via data sources associated with the patient. Still other embodiments of the present invention will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a functional block diagram showing, by way of example, an automated patient management environment.

FIG. 2 is a block diagram showing, by way of example, patient characteristics for a remotely managed patient presenting with a past or present disease state.

FIG. 3 is a block diagram showing, by way of example, classes of treatment regimens for a remotely managed patient presenting with a disease state.

FIG. 4 is a data flow diagram showing comparative population data analysis in the automated patient management environment of FIG. 1.

FIG. 5 is a flow diagram showing a method for providing goal-oriented patient management based upon comparative population data analysis, in accordance with one embodiment.

FIG. 6 is a flow diagram showing a routine for defining a therapy goal for use in the method of FIG. 5.

FIG. 7 is a flow diagram showing a routine for selecting a patient population for use in the method of FIG. 5.

FIG. 8 is a flow diagram showing a routine for identifying treatment regimens for use in the method of FIG. 5.

FIG. 9 is a flow diagram showing a routine for following a patient for use in the method of FIGURE S.

FIG. 10 is a flow diagram showing a routine for updating treatment regimens for use in the routine of FIG. 9.

FIG. 11 is a block diagram showing a system for providing goal-oriented patient management based upon comparative population data analysis, in accordance with one embodiment.

### DETAILED DESCRIPTION

Automated Patient Management Environment

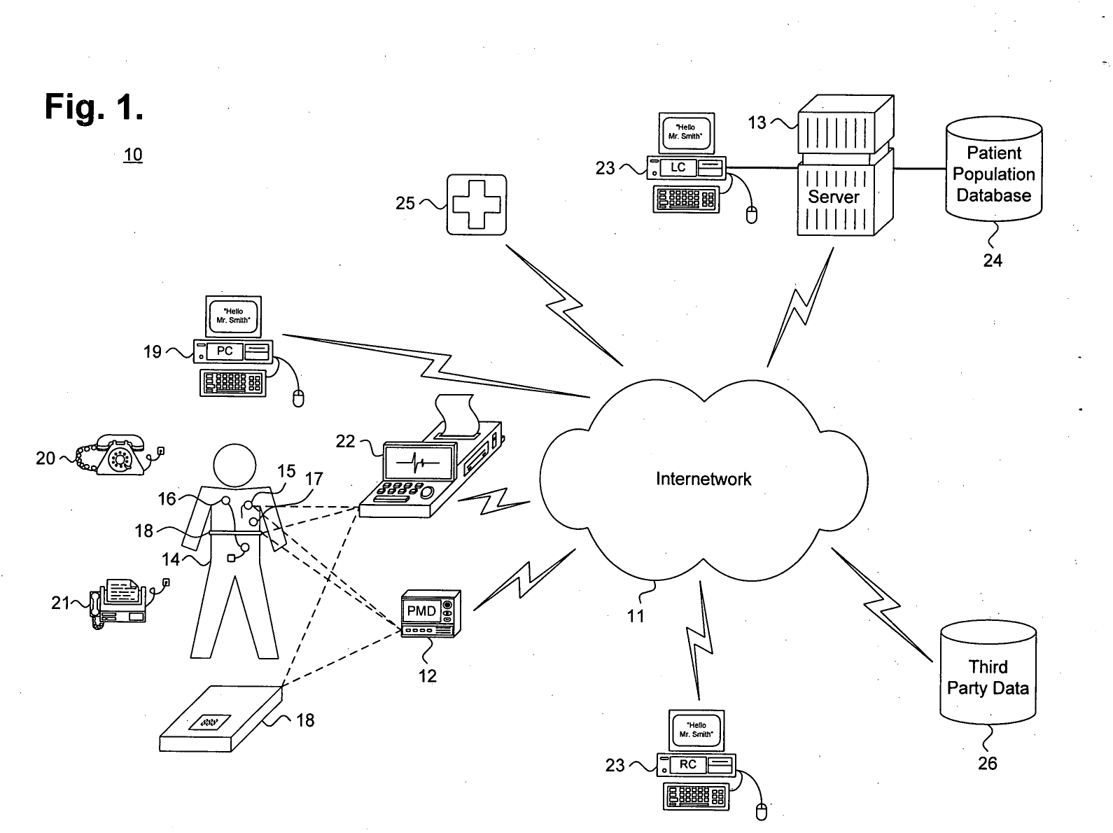

Automated patient management encompasses a range of activities, including remote patient management and automatic diagnosis of patient health, such as described in commonly-assigned U.S. Patent application Pub. No. US2004/0103001, published May 27, 2004, pending, the disclosure of which is incorporated by reference. Such activities can be performed proximal to a patient, such as in the patient's home or office, centrally through a centralized server, such from a hospital, clinic or physician's office, or through a remote workstation, such as a secure wireless mobile computing device. FIG. 1 is a functional block diagram showing, by way of example, an automated patient management environment 10. In one embodiment, a patient 14 is proximal to one or more patient monitoring or communications devices, which are interconnected remotely to a centralized server 13 over an internetwork 11, such as the Internet, or through a public telephone exchange (not shown), such as a conventional or mobile telephone network. The patient monitoring or communications devices non-exclusively include a patient management device 12, such as a repeater, personal computer 19, including a secure wireless mobile computing device, telephone 20, including a conventional or mobile telephone, and facsimile machine 21. In a further embodiment, a programmer 22, such as a programmer or programmer-recorder monitor, can be used by clinicians, such as physicians, nurses, or qualified medical specialists, to interrogate and program medical devices. Finally, the centralized server 13 is remotely interfaced to a patient care facility 25, such as a clinic or hospital, to ensure access to medical response or patient care providers. Other patient monitoring or communications devices are possible. In addition, the internetwork 11 can provide both conventional wired and wireless interconnectivity. In one embodiment, the internetwork 11 is based on the Transmission Control Protocol/Internet Protocol (TCP/IP) network communication specification, although other types or combination of networking implementations are possible. Similarly, other network topologies and arrangements are possible.

Each patient management device 12 is uniquely assigned to a patient under treatment 14 to provide a localized and network-accessible interface to one or more medical devices, which serve as patient data sources 15-18, either through direct means, such as wired connectivity, or through indirect means, such as inductive coupled telemetry, optical telemetry, or selective radio frequency or wireless telemetry based on, for example, “strong” Bluetooth or IEEE 802.11 wireless fidelity “WiFi” and “WiMax” interfacing standards. Other configurations and combinations of patient data source interfacing are possible.

Patient data includes physiological measures, which can be quantitative or qualitative, parametric data regarding the status and operational characteristics of the patient data source itself, and environmental parameters, such as the temperature, barometric pressures, or time of day. The patient data sources collect and forward the patient data either as a primary or supplemental function. Patient data sources 15-18 include, by way of example, medical therapy devices that deliver or provide therapy to the patient 14, medical sensors that sense physiological data in relation to the patient 14, and measurement devices that measure environmental parameters occurring independent of the patient 14. Other types of patient data are possible, such as third party data 26 received from external data sources, including repositories of empirical studies, public and private medical databases, patient registries, and the like. Additionally, current clinician-established guidelines associated with treatment can help to guide acceptable best practice treatment for patient care. Each patient data source can generate one or more types of patient data and can incorporate one or more components for delivering therapy, sensing physiological data, measuring environmental parameters, or a combination of functionality.

In a further embodiment, data values can be entered by a patient 14 directly into a patient data source. For example, answers to health questions could be input into a measurement device that includes interactive user interfacing means, such as a keyboard and display or microphone and speaker. Such patient-provided data values could also be collected as patient information. Additionally, measurement devices are frequently incorporated into medical therapy devices and medical sensors. Medical therapy devices include implantable medical devices (IMDs) 15, such as pacemakers, implantable cardiac defibrillators (ICDs), drug pumps, and neuro-stimulators, and external medical devices (EMDs) 16, such as automatic external defibrillators (AEDs). Medical sensors include implantable sensors 17, such as implantable heart and respiratory monitors and implantable diagnostic multi-sensor non-therapeutic devices, and external sensors 18, such as 24-hour Holter arrhythmia monitors, ECG monitors, weight scales, glucose monitors, oxygen monitors, and blood pressure monitors. Other types of medical therapy, medical sensing, and measuring devices, both implantable and external, are possible.

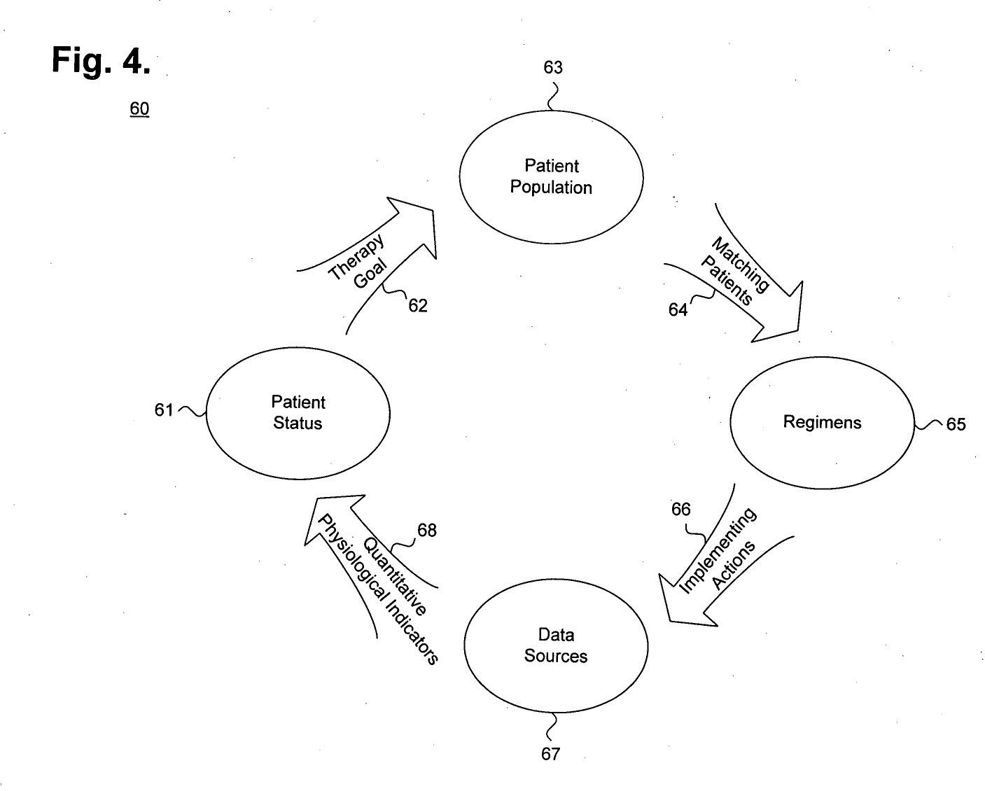

The patient management device 12 collects and temporarily stores patient data from the patient data sources 15-18 for periodic upload over the internetwork 11 to the server 13 and storage in a patient population database 24. The stored patient data can be analyzed to provide goal-oriented patient management, as further described below, beginning with reference to FIG. 4. Briefly, a clinician defines a therapy goal for a patient based on a stored physiological assessment of a diagnosed disease state. The therapy goal can be stated in broad terms, such as “treat hypertension,” which the centralized server 13 compares to the stored patient data to formulate a treatment plan that includes regimens to implement the therapy goal. New patient data received from the patient data sources 15-18 for the patient is continually evaluated to track progress toward the therapy goal.

Each patient data source 15-18 collects the quantitative physiological measures on a substantially continuous or scheduled basis and also records the occurrence of events, such as therapy or irregular readings. In a still further embodiment, the patient management device 12, personal computer 19, telephone 20, or facsimile machine 21 record or communicate qualitative quality of life (QOL) measures that reflect the subjective impression of physical well-being perceived by the patient 14 at a particular time. Other types of patient data collection, periodicity and storage are possible.

In a further embodiment, the collected patient data can also be accessed and analyzed by one or more clients 23, either locally-configured or remotely-interconnected over the internetwork 11. The clients 23 can be used, for example, by clinicians to securely access stored patient data assembled in the database 21 and to select and prioritize patients for health care provisioning, such as respectively described in commonly-assigned U.S. patent application Ser. No. 11/121,593, filed May 3, 2005, pending, and U.S. patent application Ser. No. 11/121,594, filed May 3, 2005, pending, the disclosures of which are incorporated by reference. Although described herein with reference to physicians or clinicians, the entire discussion applies equally to organizations, including hospitals, clinics, and laboratories, and other individuals or interests, such as researchers, scientists, universities, and governmental agencies, seeking access to the patient data.

In a further embodiment, patient data is safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health- and medical-related information is treated as protectable, although other types of sensitive information in addition to or in lieu of specific patient health information could also be protectable. Additionally, for purposes of utilizing information in the population database 24 or third party data 26, comparison data can be de-identified, such that specific patient identification is not available.

Preferably, the server 13 is a server-grade computing platform configured as a uni-, multi- or distributed processing system, and the clients 23 are general-purpose computing workstations, such as a personal desktop or notebook computer. In addition, the patient management device 12, server 13 and clients 23 are programmable computing devices that respectively execute software programs and include components conventionally found in computing device, such as, for example, a central processing unit (CPU), memory, network interface, persistent storage, and various components for interconnecting these components.

Patient Characteristics

The patient population database 24 contains stored patient data for a set of remotely managed patients. The patient population 24 database can also store patient data for non-remotely managed patients and from external sources, such as clinical studies. The remotely managed patients are continually monitored and the stored patient data continues to evolve and grow as patient therapies and conditions change. The patient data stored in the patient population database can be analyzed to recognize good outcomes versus bad and, if appropriate, those treatment regimens presenting a preferred path to progressing patients towards their therapy goals are identified. Additionally, the patients are individually followed by their respective clinician for one or more particular disease states for which they have been diagnosed. The patient data can be evaluated to provide goal-oriented patient management, as further described below beginning with reference to FIG. 4 et seq.

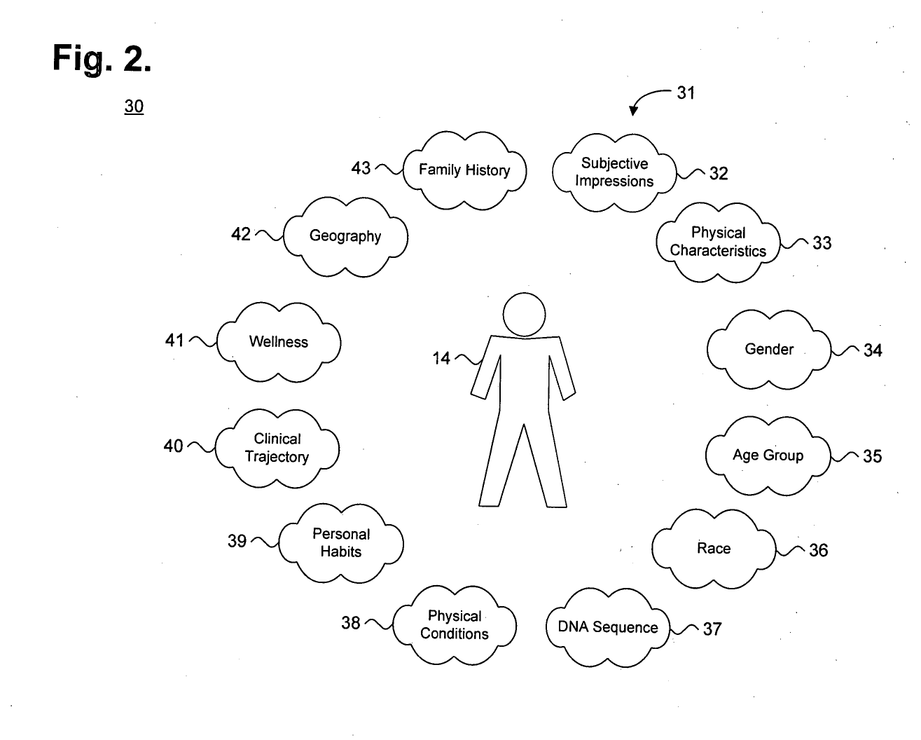

The patient population database 24 provides a data warehouse against which the characteristics and related factors of a patient under treatment can be compared to and evaluated against patient population characteristics, historical response data, outcomes, clinical trajectories, and similar information to assist with remote automated patient care. The patient data includes historical data of patients' responses to various treatment regimens and current health conditions. FIG. 2 is a block diagram 30 showing, by way of example, patient characteristics 31 for a remotely managed patient 14 presenting with -past or present disease state. The patient characteristics 31 can include both quantitative and qualitative patient information. Stable and relatively unchanging patient data, such as physical characteristics 33, gender 34, age group 35, race 36, DNA sequence 37, and geography 42, can be included in the patient population database 24 for direct comparison to the corresponding characteristics of the patient 14. Dynamic and continually changing patient characteristics 31, such as subjective health impression 32, physical conditions 38, personal habits 39, clinical trajectory 40, patient wellness 41, and family history 43, can be similarly maintained in the patient population database 24 for comparative selection of similar or matching patients presenting with the same or related disease state or co-morbidity. Additionally, patient diagnoses, such as for co-morbidities, for example, hypertension, apnea, or diabetes, or disease classification, for instance, New York Health Association classes I-IV, can be included in the patient characteristics 31. Other types of quantitative and qualitative patient, both static and dynamic, characteristics are possible.

The patient population database 24 can be organized to facilitate identifying appropriate patient subgroups. One form of organization is based upon patient characteristics. In addition, the database can be organized based on historical response data, outcomes, clinical trajectories, and similar information. As well, the patients can be grouped into subpopulations or identified individually in an anonymous de-identified fashion.

Treatment Regimens

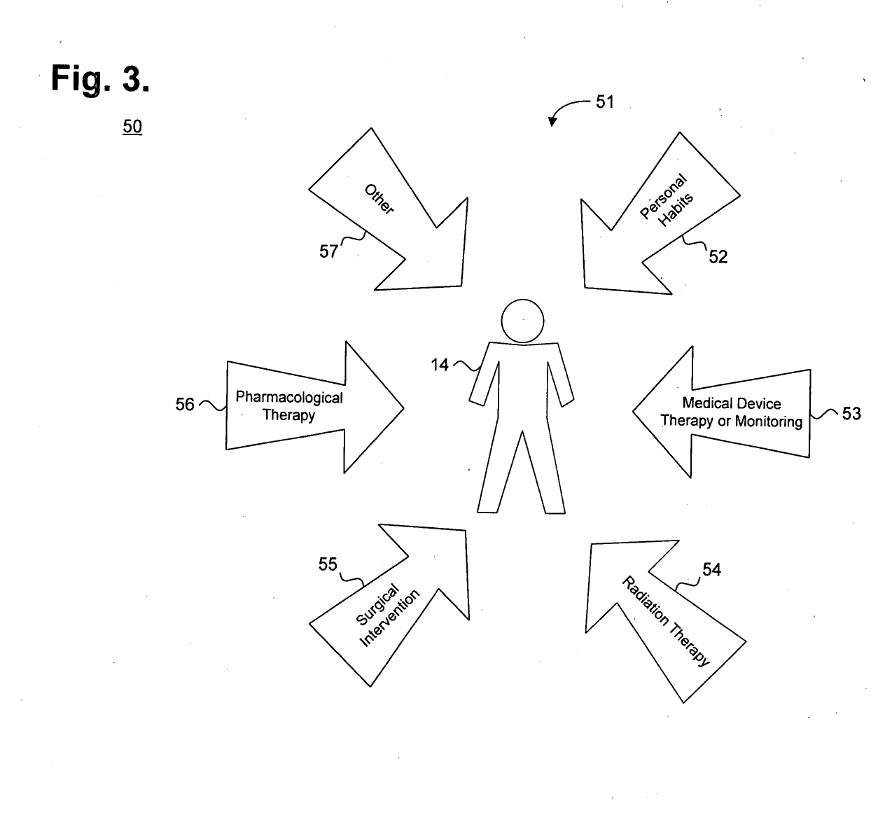

Medical care can be defined broadly to embrace almost any form of treatment regimen that could potentially be applied to other patients presenting with the same or related disease state or co-morbidity. Treatment regimens can be automatically paired with a therapy goal defined by a clinician to form a treatment plan. The pairings can be formed by evaluating a patient's current health condition and the therapy goal and looking at the historical records of patients who started out in the same or similar health condition. FIG. 3 is a block diagram 50 showing, by way of example, classes of treatment regimens 51 for a remotely managed patient 14 presenting with a disease state. The treatment regimens 51 can loosely be formed into classes, subclasses, or groups of classes of medical healthcare providing. For example, modifications to personal habits 52, such as eating a low sodium diet and exercising regularly, represent a form of informal treatment regimen 51, that fall outside of the direct control of a clinician, but nevertheless require patient compliance. Conversely, medical device therapy or monitoring 53, radiation therapy 54, surgical intervention 55, and pharmacological therapy 56, require direct clinician supervision and following and, in the case of surgical intervention 55, active involvement. Other classes, subclasses, or groups of classes and types of treatment regimens 57 are possible.

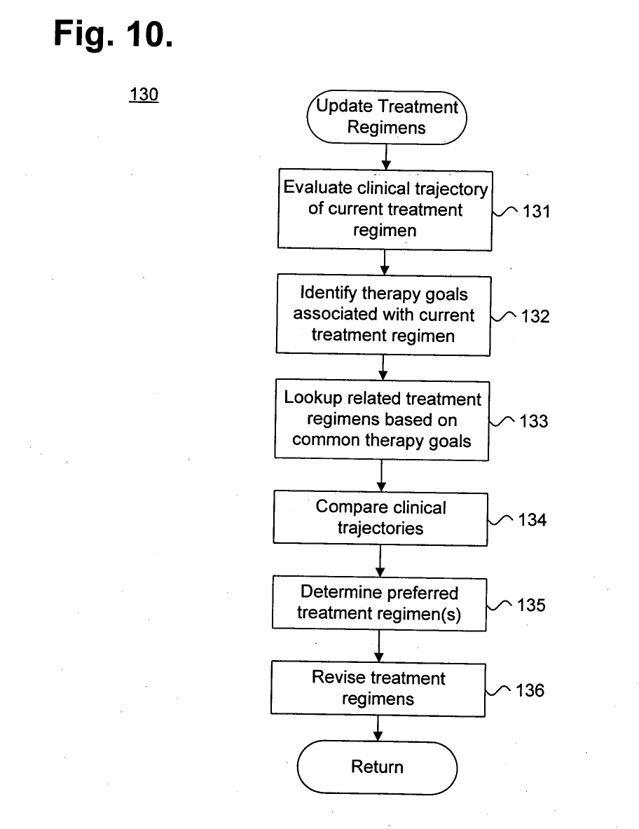

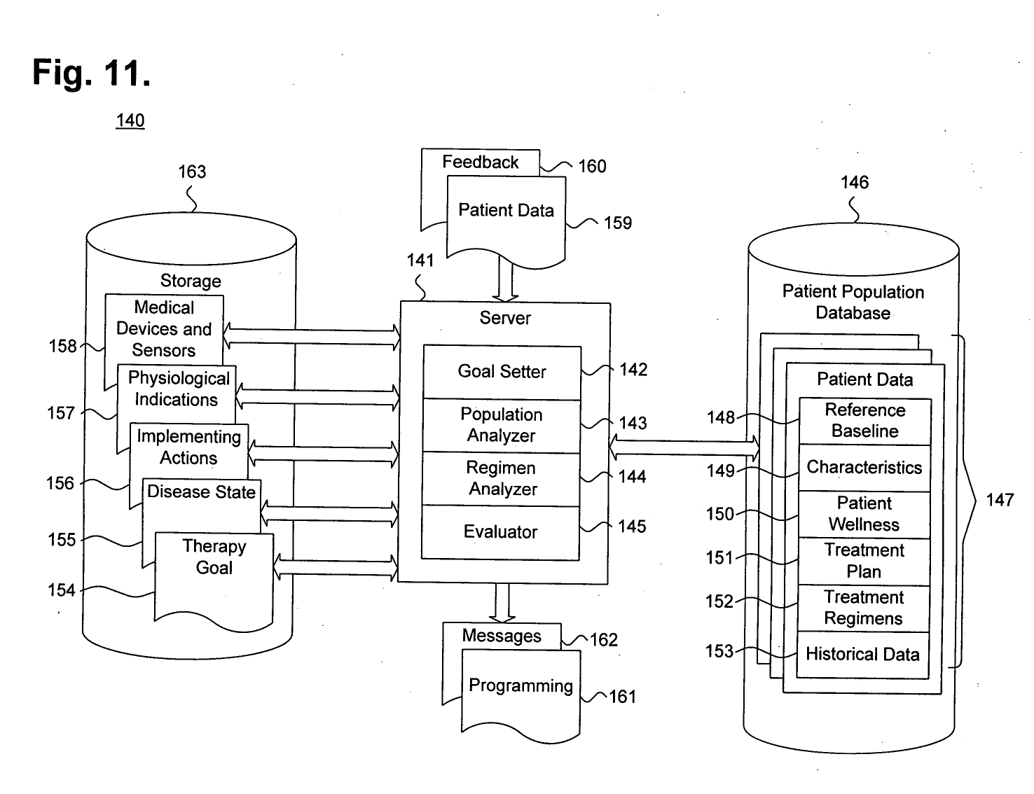

The treatment regimens 51 are included in the patient population database 24 as part of the stored patient data and provide a catalogue of possible treatment strategies for a particular disease state as applied by various clinicians across the spectrum of patients in the patient population. A treatment plan can be formulated by selecting those treatment regimens 51 that are associated with patients in the patient population sharing at least one characteristic or related factor with the patient under treatment and who started out in the same or similar health condition. The treatment plans are compared within the database to determine good or, if possible, preferred treatment regimens based on an evaluation of clinical trajectories for common therapy goals, as further described below with reference to FIG. 10. Each particular treatment regimen 51 can become an implementing action that would be applied to or undertaken by the patient under treatment to progress towards a therapy goal. For example, a therapy goal to treat hypertension could be implemented by undertaking treatment regimens 51 that can include prescribing diuretics and vasodilators, adopting a low sodium and low saturated fat diet, performing regular exercise, and ceasing smoking, if applicable.

Patients' compliance with the treatment plan can be followed and remotely monitored by following quantitative physiological indications, such as blood pressure, weight, and heart rate. In a further embodiment, qualitative physiological indications can also be followed, such as by obtaining quality of life measures. A quality of life measure is a semi-quantitative self-assessment of an individual patient's physical and emotional well-being and a record of symptoms, such as provided by the Duke Activities Status Indicator. Other qualitative and quality of life measures are possible, such as those indicated by responses to the Minnesota Living with Heart Failure Questionnaire described in E. Braunwald, ed., “Heart Disease-A Textbook of Cardiovascular Medicine,” pp. 452-454, W.B. Saunders Co. (1997), the disclosure of which is incorporated by reference. Similarly, functional classifications based on the relationship between symptoms and the amount of effort required to provoke them can serve as quality of life and symptom measures, such as the NYHA classifications I-IV, also described in Ibid. As necessary, a clinician can also review and follow the implementing actions nominated under a treatment plan for appropriateness and patient safety.

Data Flow

The stored patient data in the patient population database 24 can be mined to identify possible treatment regimens for a patient presenting with indications of a particular or related disease state or co-morbidity matched with at least one other patient in the patient population database 24. FIG. 4 is a data flow diagram 60 showing comparative population data analysis in the automated patient management environment 10 of FIG. 1. Data analysis is performed for a patient presenting with indications of a diagnosed disease state as part of an automated iterative process that includes closed loop assessment and following. In a further embodiment, the automated iterative process can include open loop assessment and following, or a combination of open and closed loop assessment and following.

Initially, a determination of the patients' current health condition and status 61 is performed and a therapy goal 62 is defined by a clinician. Matching patients 64 are then selected out of the patient population 63 by identifying one or more patient characteristics shared with the patient under treatment to find appropriate treatment regimens 65. The treatment regimens 65 are evaluated by first looking at the historical records for patients that started out in the same or similar health condition as the patient under treatment and identifying the treatment regimens most likely to succeed with the least harm to the patient. Those treatment regimens acceptable with the patients' current health condition are identified and, if available, a preferred path is selected. Shared patient characteristics can include, for instance, physical characteristics, such as gender, ethnicity, age group, and stature, and health conditions, including the same or similar disease state or co-morbidity, plus other considerations. Other forms of population-based comparison and matching are widely known and practiced and could apply equally in identifying the matching patients 64.

Similarly, the implementing actions 66 can be widely grouped to include a spectrum of medical healthcare providing, from prescribed and closely monitored medical treatments to more informal forms of healthcare, such as patient habit or behavior modifications. Preferably, the implementing actions 66 are capable of being remotely managed. Similarly, implementing actions 66 are identified from the treatment regimens 65 associated with the matching patients 64. The set of implementing actions 66 for a given patient form a treatment plan to implement a therapy goal as specified by a clinician.

In a further embodiment, the set of implementing actions 66 are provided to the clinician as a recommendation and can require express approval and following before being executed or undertaken by the patient under treatment. The treatment regimens 65 can be pre-classified and presented under the treatment plan in ranked order, which the clinician can review and approve. Other forms of treatment plan formulation are possible.

Once the treatment plan has been effected by the patient under treatment by executing or undertaking the set of implementing actions 66, quantitative physiological indications 68 are followed by monitoring the data sources 67 associated with the patient. In a further embodiment, qualitative physiological indicators are followed in addition to or in lieu of the quantitative physiological indicators. A patient status 61 is periodically generated based on the therapy goal 62, which is used to evaluate the patient and, if necessary, reassess the treatment plan to better address the needs of the patient based on both the patient status 61 and new patient data obtained from the patient population database 24. The clinical trajectories are evaluated to identify good or, if possible, preferred trajectories over bad. Other forms and types of processing and data handling are possible.

Method Overview

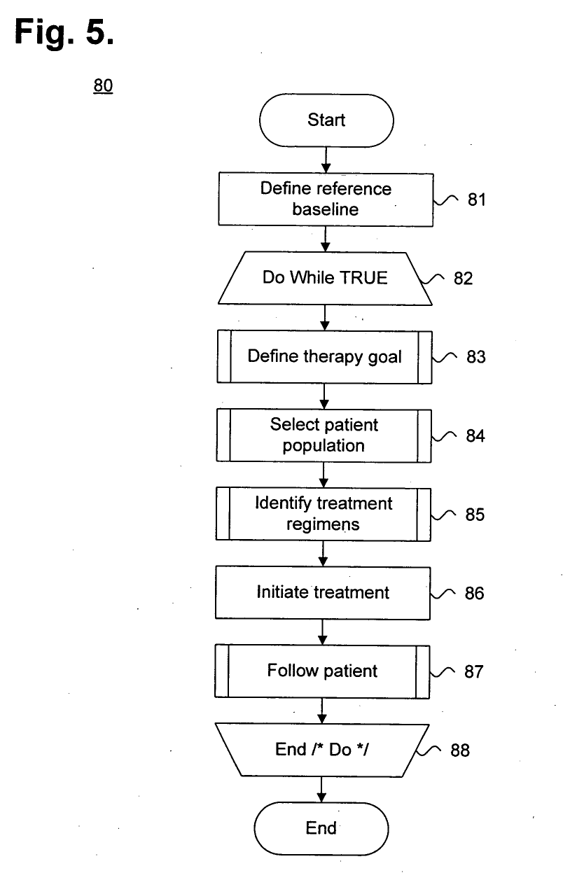

Goal-oriented patient management is performed continuously in a closed loop by cycling through a data mining analysis of the patient population database 24 and the healthcare condition of the patient under treatment, as appropriate. FIG. 5 is a flow diagram showing a method 80 for providing goal-oriented patient management based upon comparative population data analysis, in accordance with one embodiment.

Initially, a reference baseline is defined for the patient under treatment during an initial observation period (block 81). The reference baseline establishes an initial patient status 61 and subsequent sets of patient data can enable the physical well-being of the patient under treatment to be followed remotely. During the initial observation, the patient under treatment performs a pattern of physical stressors and an initial set of quantitative physiological measures and, in a further embodiment, qualitative physiological measures, are generated and stored as part of the patient profile.

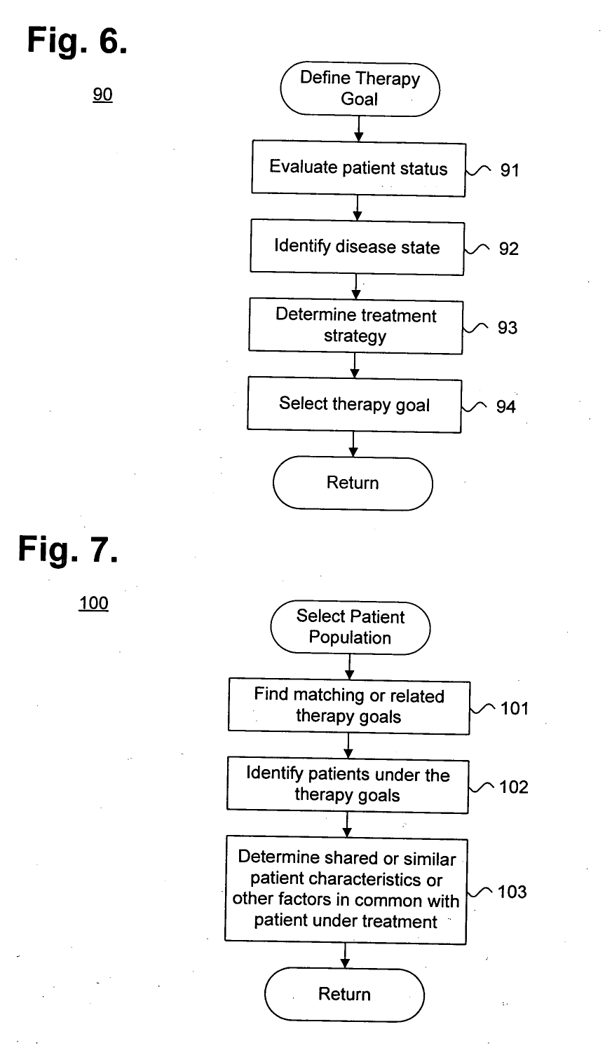

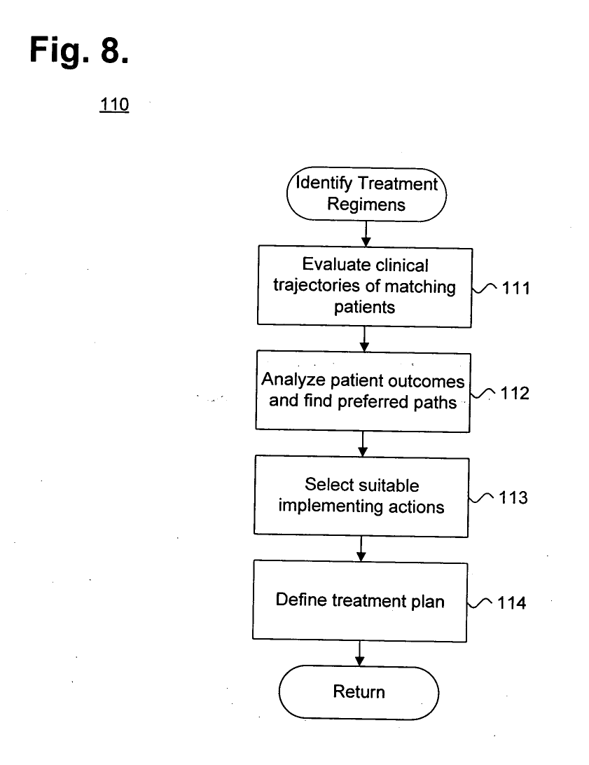

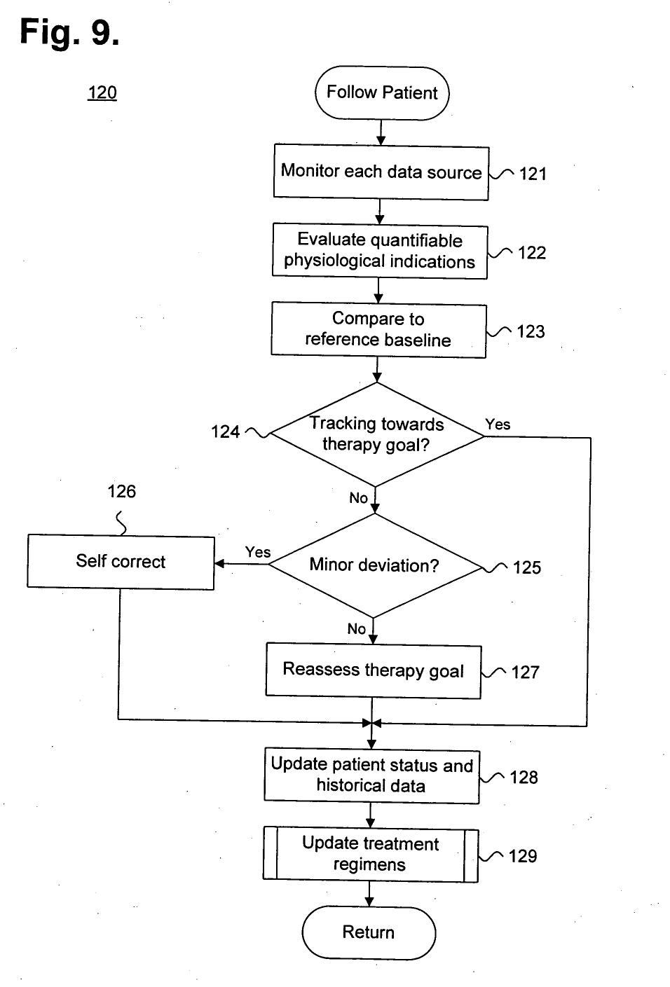

Subsequently, the method 80 proceeds by managing the patient in a continuous closed loop cycle (blocks 82-88). During each cycle (block 82), one or more therapy goals can be defined by a clinician (block 83), as further described below with reference to FIG. 6. An initial therapy goal must be defined and can be modified or replaced as necessary during subsequent cycles. To form each therapy goal, a patient population matching the patient under treatment based on one or more patient characteristics is selected (block 84) and treatment regimens are identified as implementing actions in a treatment plan (block 85), as further described below respectively with reference to FIGS. 7 and 8. The treatment plan is then initiated (block 86) and the patient is followed (block 87), as further described below with reference to FIG. 9.

In a further embodiment, the treatment plan is presented to a clinician for review and approval prior to being initiated and can also be manually followed by the clinician in an open loop cycle. Combinations of closed and open loop cycles are possible. Processing continues (block 88) until the processing infrastructure, for instance,,the centralized server 13, terminates execution.

Therapy Goal Definition

A therapy goal must first be specified by a clinician, but details for implementing the therapy goal are formulated as a treatment plan automatically generated by the centralized server 13 based on a comparative analysis of the patient data in the patient population database 24. FIG. 6 is a flow diagram showing a routine 90 for defining a therapy goal for use in the method 80 of FIG. 5.

Initially, the wellness status of the patient is evaluated (block 91) and a disease state is identified (block 92), such as described in related, commonly-owned U.S. Pat. No. 6,336,903, to Bardy, issued Jan. 8, 2002; U.S. Pat. No. 6,368,284, to Bardy, issued Apr. 9, 2002; U.S. Pat. No. 6,398,728, to Bardy, issued Jun. 2, 2002; U.S. Pat. No. 6,411,840, to Bardy, issued Jun. 25, 2002; and U.S. Pat. No. 6,440,066, to Bardy, issued Aug. 27, 2002, the disclosures of which are incorporated by reference. A treatment strategy is then outlined (block 93) and a therapy goal is selected (block 94). In one embodiment, the treatment strategy outlines the overall healthcare needs of the patient, while the therapy goal seeks to address a specific health condition. For example, a treatment strategy for preventative cardiac management might include controlling hypertension as a therapy goal. Other types of treatment strategies and therapy goals are possible.

Patient Population Selection

A patient population can be selected based on one or more patient characteristics or related factors shared with the patient under treatment. The patient population can include a class, subclass, or group of classes. FIG. 7 is a flow diagram showing a routine 100 for selecting a patient population for use in the method 80 of FIG. 5. In addition to patient characteristics, the patient population can be selected based on historical response data, outcomes, clinical trajectories, and similar considerations. The patient population database 24 is organized to facilitate identifying patient populations.

Initially, the patient population database 24 is searched to find matching or related therapy goals (block 101). Those patients treated at some point under the matching therapy goals are identified (block 102). Shared or similar patient characteristics in common with the patient under treatment are determined with those patients having the best fit being assigned into the patient population (block 103).

Treatment Regimen Identification

The treatment regimens undertaken by those patients in the patient populations matching the patient under treatment form the basis for implementing actions in a treatment plan under the therapy goal. Those treatment regimens resulting in good or, if possible, preferred outcomes are weighted more heavily than those regimens that result in bad outcomes. FIG. 8 is a flow diagram showing a routine 110 for identifying treatment regimens for use in the method 80 of FIG. 5. Where available, a preferred path to progressing the patient towards the therapy goal is identified. The patient population database 24 is organized to facilitate identifying treatment regimens.

Initially, the clinical trajectories of the matching patients are evaluated (block 111). Evaluation of the clinical trajectories is critical to ensuring that each matching patient is trending in a direction that is consistent with the therapy goal for the patient under treatment. The patient outcomes are analyzed and, where available, a preferred path to progress the patient under treatment towards the therapy goal is found (block 112). Based on the therapy regimen required to produce a favorable patient outcome, appropriate implementing actions are selected (block 113). The implementing actions can include or omit those implementing actions performed under the identified treatment regimens as necessary to compensate for the health condition of the patient under treatment. The selected implementing actions define a treatment plan (block 114), which, in a further embodiment, can be further compared to the treatment plans applied to past patients to help ensure the selection of a treatment plan most likely to succeed and causing the least harm to the patient. Other types of treatment regimen identifications and refinements are possible.

Patient Following

Once initiated, the implementing actions are followed on a continuing or periodic basis, depending upon the types and degrees of implementing actions. FIG. 9 is a flow diagram showing a routine 120 for following a patient for use in the method 80 of FIG. 5. In addition, changes to the clinical trajectory of the patient under treatment due to the treatment regimens applied are reflected in the patient population database 24.

The following of a patient under treatment can be performed remotely by monitoring each patient data source 15-18 for the patient under treatment (block 121), such as described in commonly-assigned U.S. Patent application, entitled “System And Method For Providing Hierarchical Medical Device Control For Automated Patient Management,” Ser. No. ______, filed on Jan. 19, 2006, pending, the disclosure of which is incorporated by reference. Quantifiable physiological indications are evaluated (block 122) for comparison to threshold and other relative or absolute measures of patient wellness indicating an onset, absence, progression, regression, or status quo of the disease state. In a further embodiment, qualitative physiological indications can also be evaluated. As necessary, the qualitative physiological indications are compared to the reference baseline (block 123). If the patient under treatment is tracking towards the therapy goal (block 124), the patient status is merely updated (block 128). Otherwise, if a minor deviation from the therapy goal presents (block 125), in a further embodiment, the implementing actions are self-corrected to address the minor deviation (block 126). Otherwise, the therapy goal is reassessed (block 127) and the patient status and historical data are updated (block 128). Finally, the treatment regimens maintained as historical patient responses in the patient population database 24 are updated (block 129), as further described below with reference to FIG. 10. Other forms of patient following, both automated and manual, are possible.

Treatment Regimens Updating

A therapy goal can be reached through one or more treatment regimens, but every treatment regimen may not necessarily lead to a favorable patient outcome. FIG. 10 is a flow diagram showing a routine 130 for updating treatment regimens 51 for use in the routine 120 of FIG. 9. The patient population database 24 organizes the treatment regimens to facilitate automated identification of those regimens that are most likely to succeed and which would least likely result in harm to the patient.

Initially, the clinical trajectory of the patient under treatment for the current treatment regimen is evaluated to determine whether the trajectory is trending towards a good, bad, or indeterminate outcome (block 131). The therapy goals associated with the current treatment regimen are identified (block 132). One or more therapy goals can be associated with a particular treatment regimen 51. Related treatment regimens based on common therapy goals are looked up in the patient population database 24 (block 133) and each respective clinical trajectory is compared (block 134). Any preferred treatment regimens are determined (block 135) and revised (block 136). Where possible, preferred paths to progressing a patient towards therapy goals are identified.

System Overview

Generally, the centralized server is responsible for managing patients through continual closed loop patient data analysis, although, in a further embodiment, the processing can be delegated to individual clients or patient management devices. FIG. 10 is a block diagram showing a system 140 for providing goal-oriented patient management based upon comparative population data analysis, in accordance with one embodiment. A server 141 implements the system 140 and executes a sequence of programmed process steps, such as described above beginning with reference to FIG. 5 et seq., implemented, for instance, on a programmed digital computer system.

The server 141 includes a goal setter 142, population analyzer 143, regimen analyzer 144, and evaluator 145. The server 141 also maintains an interface to the patient population database 146 and storage 163. The patient population database 146 is used to maintain patient data 147, which can include a reference baseline 148, characteristics 149, patient wellness 150, treatment plan 151, treatment regimens 152, and historical data 153. The patient wellness 150 and historical data 153 respectively reflect the current and past health conditions of the patient, including responses to treatment based on therapy goals. Other types of patient information are possible. The patient information 147 is maintained for those patients belonging to the population of patients managed by the server 141, as well as for other patients not strictly within the immediate patient population, such as retrieved from third party data sources.

The storage 163 is used to maintain listings of the medical devices and sensors 158 managed by the patient management devices 12 and any programmers or similar devices 22 that can interrogate or program the medical devices or sensors 158. The storage 163 also includes a set of implementing actions 156 and physiological indications 157 for each patient under treatment. The physiological indications 157 are generated by the data sources associated with the patient under treatment during monitoring and can include quantitative and, in a further embodiment, qualitative physiological measures. The implementing actions 156 provide a treatment plan 151 to move the patient towards a therapy goal 154 based on a diagnosed disease state 155. Other types of device information, implementing actions, quantifiable physiological indications, therapy goals, disease states, and other condition management information are possible.

The goal setter 142 is used by a clinician to define the therapy goal 154 based on the wellness 150 of the patient under treatment, available medical devices and sensors 158, any preferences of the clinician, and other factors that can be expressed in a general but implementable form. The goal setter 142 might be used to specify the therapy goal 154 based on an overall treatment strategy or on an indication for a specific type of healthcare. Other goal setting approaches are possible.

The goal setter 142 operates in conjunction with the population analyzer 143 and regimen analyzer 144 to formulate the treatment plan 151. The population analyzer 143 evaluates the patient data 147 maintained in the patient population database 146 to identify those patients with matching or similar characteristics 149 or other factors of the patient under treatment. Other factors include historical response data, outcomes, and clinical trajectories. The patients are selected by matching or related therapy goals. Shared or similar patient characteristics or other factors in common with the patient under treatment are determined. Similarly, the regimen analyzer 144 identifies those treatment regimens 152 appropriate for the patient. The treatment regimens 152 are selected by evaluating the clinical trajectories of the matching patients and analyzing the patient outcomes to find, if available, any preferred paths for treatment. The outputs of the population analyzer 143 and regimen analyzer 144 are used by the goal setter 142 to formulate the set of implementing actions 156 for the patient under treatment.

Finally, the evaluator 145 initiates the treatment plan by dispatching programming 161 and, as necessary, messages 162 to execute the implementing actions 156. In a further embodiment, the evaluator 145 also generates a reference baseline 148 of patient well-being for the patient under treatment. Alternatively, the reference baseline could be provided by a separate data source. The evaluator 145 receives updated patient data 159 and feedback 160 to track the physiological indications 157 for the patient under treatment in response to the execution of the implementing actions 156. Other components and functionality are possible.

While the invention has been particularly shown and described as referenced to the embodiments thereof, those skilled in the art will understand that the foregoing and other changes in form and detail may be made therein without departing from the spirit and scope of the invention.

What is claimed is:

1. A system for providing goal-oriented patient management based upon comparative population data analysis, comprising:
a goal setter to define at least one therapy goal to manage a disease state, comprising:
a patient population selected to share at least one characteristic with an individual patient presenting with indications of the disease state; and
one or more treatment regimens associated with the patient population identified as implementing actions under the at least one therapy goal;
an evaluator to follow the implementing actions through one or more quantifiable physiological indications monitored via data sources associated with the patient.

2. A system according to claim 1, further comprising:
a reference baseline assessed for the individual patient comprising one or more physiological measures which each relate to patient data recorded during an initial time period.

3. A system according to claim 2, wherein the reference baseline is reassessed for the individual patient based on patient data obtained from the one or more quantifiable physiological indications.

4. A system according to claim 1, wherein at least one of the implementing actions are actively monitored for the individual patient via the data sources.

5. A system according to claim 1, wherein the implementing actions are followed through one or more qualitative physiological indications monitored via the data sources.

6. A system according to claim 1, wherein the goal setter provides the therapy plan as a recommendation to a clinician and implements the therapy plan upon instructions from the clinician.

7. A system according to claim 1, wherein the patient characteristics are selected from the group comprising subjective impressions, physical characteristics, gender, age group, race, DNA sequence, physical conditions, personal habits, clinical trajectory, wellness, geography, and family history.

8. A system according to claim 1, wherein the treatment regimens are selected from the group of classes comprising personal habit modification, medical device therapy or monitoring, radiation therapy, surgical intervention, and pharmacological therapy.

9. A system according to claim 1, wherein the data sources are selected from the group comprising implantable medical devices, external medical devices, implantable sensors, and external sensors.

10. A method for providing goal-oriented patient management based upon comparative population data analysis, comprising:
defining at least one therapy goal to manage a disease state, comprising:
selecting a patient population sharing at least one characteristic with an individual patient presenting with indications of the disease state; and
identifying one or more treatment regimens associated with the patient population as implementing actions under the at least one therapy goal;
following the implementing actions through one or more quantifiable physiological indications monitored via data sources associated with the patient.

11. A method according to claim 10, further comprising:
assessing a reference baseline for the individual patient comprising one or more physiological measures which each relate to patient data recorded during an initial time period.

12. A method according to claim 11, further comprising:
reassessing the reference baseline for the individual patient based on patient data obtained from the one or more quantifiable physiological indications.

13. A method according to claim 10, further comprising:
actively monitoring at least one of the implementing actions for the individual patient via the data sources.

14. A method according to claim 10, further comprising:
following the implementing actions through one or more qualitative physiological indications monitored via the data sources.

15. A method according to claim 10, further comprising:
providing the therapy plan as a recommendation to a clinician; and
implementing the therapy plan upon instructions from the clinician.

16. A method according to claim 10, wherein the patient characteristics are selected from the group comprising subjective impressions, physical characteristics, gender, age group, race, DNA sequence, physical conditions, personal habits, clinical trajectory, wellness, geography, and family history.

17. A method according to claim 10, wherein the treatment regimens are selected from the group of classes comprising personal habit modification, medical device therapy or monitoring, radiation therapy, surgical intervention, and pharmacological therapy.

18. A method according to claim 10, wherein the data sources are selected from the group comprising implantable medical devices, external medical devices, implantable sensors, and external sensors.

19. A computer-readable storage medium holding code for performing the method according to claim 10.

20. An apparatus for providing goal-oriented patient management based upon comparative population data analysis, comprising:
means for defining at least one therapy goal to manage a disease state, comprising:
means for selecting a patient population sharing at least one characteristic with an individual patient presenting with indications of the disease state; and
means for identifying one or more treatment regimens associated with the patient population as implementing actions under the at least one therapy goal;
means for following the implementing actions through one or more quantifiable physiological indications monitored via data sources associated with the patient.
